(19) United States
(12) Patent Application Publication
(10) Pub. No.: US 2023/0084959 A1
(43) Pub. Date: Mar. 16, 2023

(54) Blood Pack Donation System for Biomarker Collection During Whole Blood Donation

(71) Applicant: Fenwal, Inc., Lake Zurich, IL (US)

(72) Inventor: James Madsen, Chicago, IL (US)

(21) Appl. No.: 17/897,475

(22) Filed: Aug. 29, 2022

Related U.S. Application Data
(60) Provisional application No. 63/241,390, filed on Sep. 7, 2021.

(51) Int. Cl. B01L 3/00; A61B 5/15; A61J 1/14; A61J 1/10

(52) U.S. Cl. CPC B01L 3/502753; B01L 3/502761; B01L 3/505; A61B 5/150366; A61J 1/1475; A61B 5/150992; A61J 1/10; B01L 2200/0652; B01L 2300/0681; B01L 2200/026; B01L 2300/0864; B01L 2400/0644; B01L 2300/0861

(57) ABSTRACT

A blood pack donation system configured for use with a lab-on-a-chip device for biomarker collection during whole blood donation including a blood collection container, a biomarker collection container, a first flow path connected to an opening in the blood collection container and to a first outlet opening of a lab-on-a-chip device, a second flow path connected to an opening in the biomarker collection container and to a second outlet opening of the lab-on-a-chip device, and a third flow path connected to a needle and to an inlet opening of the lab-on-a-chip device. The system may be used in a single pass collection procedure. A second version includes a fourth flow path connected to the first flow path and to the third flow path, with a plurality of flow control components that selectively control flow to provide a single pass collection procedure or a multiple pass collection procedure.

### BACKGROUND

### Field of the Disclosure

The invention relates to blood collection systems. More particularly, the invention relates to devices used during whole blood donation, and devices for collection of biomarkers.

### Description of Related Art

Whole blood (hereinafter “WB”) donation procedures typically are conducted with the sole purposed of WB collection. However, there is a rise in development of new in vitro diagnostics (hereinafter “IVD”), which are tests done on samples, such as blood or tissue, taken from a human subject. IVD can detect diseases or other conditions and may be used to monitor overall health and to potentially to help cure, treat or prevent diseases. IVD also can be used in precision medicine to identify patients who are likely to benefit from specific treatments or therapies.

Lab-on-a-chip (hereinafter “LOC”) devices are commonly used in IVD. Such devices integrate one or several laboratory functions on a single integrated circuit (or chip) to achieve automation and high-throughput screening. LOC devices can handle extremely small fluid volumes, down to less than pico-liters.

LOC technology may soon become an important part of efforts to improve global health, particularly through the development of point-of-care (hereinafter “POC”) testing devices. In countries with few healthcare resources, infectious diseases that would be treatable in a developed nation are often deadly. In some cases, poor healthcare clinics have the drugs to treat a certain illness, but lack the diagnostic tools to identify patients who should receive the drugs. Many researchers believe that LOC technology may be a key to powerful new diagnostic instruments. The goal of these researchers is to create microfluidic chips that will allow healthcare providers to perform diagnostic tests such as immunoassays and nucleic acid assays with no laboratory support. Development of LOG devices may provide numerous advantages, which are specific to theft application.

Within the field of IVD, small volumes of WB may be collected from patients for analysis of circulating biomarkers. The analysis of plasma biomarkers can diagnose many diseases, such as cancer, Alzheimer's disease, or sepsis. Typically, plasma is separated from WB before analysis to prevent contamination of the biomarkers by the presence of leukocytes, erythrocytes and hemolysis, which could increase test variability and reduce test accuracy.

The current state of the art for acquiring plasma for LOC devices consists of either using a traditional bench-top centrifuge or using plasma separation filters. In turn, each company developing a LOC device also develops its own in-vitro diagnostic device to collect biomarkers on a disposable cartridge. These separate processes and devices unfortunately do not lend themselves to point-of-care devices for wide-spread use or use during routine WB donations.

### SUMMARY

There are several aspects of the present subject matter which may be embodied separately or together in the devices and systems described and claimed herein. These aspects may be employed alone or in combination with other aspects of the subject matter described herein, and the description of these aspects together is not intended to preclude the use of these aspects separately, or the claiming of such aspects separately or in different combinations as set forth in the claims appended hereto.

The prospect of direct biomarker collection during WB donation could provide significant advantages in costs, efficiencies and timeliness. This may be facilitated by providing for use of LOC devices that provide for separation of biomarkers during WB donation, which will be the context for use of “LOC device(s)” hereinafter.

This disclosure seeks to provide a standardized blood pack donation system configured for use with a LOC device for biomarker separation and collection during WB donation. Many biotechnology companies are working to develop such LOC devices to separate, collect and/or detect biomarkers for diseases. These LOC devices typically require low flow rates as biomarkers are separated from WB. The invention of the present disclosure would provide a standardized blood pack donation system or kit, which would allow for LOC device testing during a routine blood donation procedure, such as for example a 500 ml WB donation. Thus, a blood pack device and methods of using the same of the present disclosure permit WB donation and facilitate simultaneous use of a LOC device for separation and collection of biomarkers for further analysis.

It will be appreciated that the blood pack donation systems disclosed herein are configured for use with any of a variety of LOC devices, which may be provided by various manufacturers for use in separation, collection and screening for any one or more of a number of different biomarkers. Thus, makers of LOC devices may integrate their respective devices for use in testing in blood donation centers, clinics, medical facilities or other locations where routine WB donation may be conducted. As such, WB donors would have an option of being screened for disease marking biomarkers during a routine WB donation procedure, such as for example a 500 ml WB donation. This would provide much broader access to biomarker screening and early disease detection for routine WB donors.

The availability of such an advantageous standardized blood pack donation system or kit for use with LOC devices in WB donation centers or other facilities would readily and conveniently enable screening many donors for disease biomarkers, while reducing costs and the logistics involved in developing and stocking individual systems from each manufacturer. It will be appreciated that this would be important to early diagnosis, prevention and treatment of cancer or any number of different diseases. A standardized system or kit may be coupled with a variety of LOC devices to yield many applications for any number of biomarkers. The convenience could prompt offers by blood donation centers, clinics or medical facilities to donate WB and screen for particular biomarkers, including but not limited to genetic material, cell free DNA, exosomes, or any particular bloodborne biomarker for a disease.

The system or kit may take advantage of microfluidics in line to the WB collection container. For example, flow may be driven via the donor's blood pressure. The WB collection container also may be relocated to hang at a greater height to reverse flow and return WB to the donor, if desired, such as during a multiple pass collection procedure. The potential to collect any biomarker by using a microfluidic or LOC device is quite broad. Thus, in a single pass collection procedure, depending on the configuration of the system, at least some of the WB that is collected during donation is run through a LOC device for separation, before reaching a WB collection container.

The WB collection container may have any configuration and selected volume and, when combined into the system, must have at least one opening. Thus, the opening may be preexisting and connected to a flow path, or may be formed when the WB collection container is being connected to a flow path within the system. In turn, the biomarkers separated out by the LOC device are collected and transferred to a biomarker collection container. The biomarker collection container also may have any configuration, such as a bag, tube, syringe or other suitable structure, and any selected volume, but generally will be much smaller than the WB collection container. When combined into the system, the biomarker collection container also must have at least one opening. Thus, the opening may be preexisting and connected to a flow path, or may be formed when the biomarker collection container is being connected to a flow path within the system.

An alternative second example system may be provided to utilize a single pass collection procedure or a multiple pass collection procedure. The second example system includes a bypass line or flow path between the donor and WB collection container. This permits procedures in which some, none or all of the WB bypasses the LOC device. The second example system may be employed in a multiple pass collection procedure, such as may include three steps. In the first step (step one), flow through the LOC device is permitted and flow through the bypass line or flow path may range from being fully or partially permitted to being prevented, depending on the portion of the donated WB intended to pass through the LOC device. Accordingly, at least some of the WB that is collected during donation from a donor is run through a LOC device for separation, before reaching a WB collection container. In turn, the biomarkers separated out by the LOC device are collected and transferred via a separate flow path to a biomarker collection container.

In a second step (step two) of the multiple pass collection procedure, flow is reversed from the WB collection container and all of the flow is permitted through the bypass line or flow path, while being entirely prevented from flowing back through the LOC device and thereby to the biomarker collection container. In a third step (step three), at least some of the WB that is collected during donation from a donor again is run through a LOC device for separation, before reaching the WB collection container. As with the first step, flow through the bypass line or flow path may range from being fully or partially permitted to being prevented. WB is collected again and at least some of the WB that is collected is run through the LOC device before reaching the WB collection container. The biomarkers separated out by the LOC device are collected and transferred to the biomarker collection container. Thus, upon completion of a multiple pass collection procedure, donated WB has been collected in the WB collection container, while biomarkers have been collected in the biomarker collection container.

In a first aspect, a blood pack donation system is configured for use with a LOC device for biomarker collection during WB donation including a blood collection container having a volume and at least one opening, a biomarker collection container having a volume and at least one opening, a first flow path having a first end connected to the opening in the blood collection container and a second end configured to be connected to a first outlet opening of the LOC device, a second flow path having a first end connected to the opening in the biomarker collection container and a second end configured to be connected to a second outlet opening of the LOC device, and a third flow path having a first end connected to a needle and a second end configured to be connected to an inlet opening of the LOC device. These components are present in first and second example systems, both of which are capable of being used in a single pass collection procedure.

In a second aspect, a blood pack donation system of the previous configuration may further include a fourth flow path having a first end connected to the first flow path at a location between the first end and second end of the first flow path and having a second end connected to the third flow path at a location between the first end and second end of the third flow path. The fourth flow path serves as the aforementioned bypass line or flow path between the donor and WB collection container, which permits bypassing the LOC device. The blood pack donation system also may include a plurality of flow control components that selectively control flow through the first flow path, third flow path and fourth flow path. The plurality of flow control components is selectively adjustable for use with a LOC device to provide a single pass collection procedure or a multiple pass collection procedure for collecting WB during a WB donation, while also collecting biomarkers. The multiple pass collection procedure includes a succession of adjustments of the plurality of flow control components.

It will be appreciated that a blood pack donation system configured for use with a LOC device for biomarker collection during WB donation may be constructed for use in advantageous, cost efficient and convenient single or multiple pass collection methods.

### BRIEF DESCRIPTION OF THE DRAWINGS

FIG. 1 is a diagram of a first example blood pack donation system for use with a LOC device for biomarker collection during WB donation utilizing single pass collection.

FIG. 2 is a diagram of a second example blood pack donation system for use with a LOC device for biomarker collection during WB donation utilizing single pass collection or multiple pass collection and illustrating flow control component settings for a single pass collection or a first step of a multiple pass collection.

FIG. 3 is a diagram showing the system in FIG. 2 and illustrating flow control component settings for a second step of a multiple pass collection.

FIG. 4 is a diagram showing the system in FIG. 2 and illustrating flow control component settings for a third step of a multiple pass collection.

### DETAILED DESCRIPTION

The embodiments disclosed herein are for the purpose of providing an exemplary description of the present subject matter. They are, however, only exemplary, and the present subject matter may be embodied in various forms. Therefore, specific details disclosed herein are not to be interpreted as limiting the subject matter as defined in the accompanying claims.

It will be appreciated that a blood pack donation system configured for use with a LOC device for biomarker collection during WB donation may be applicable for efficient and convenient use with a LOC device to screen for the presence of and collect biomarkers.

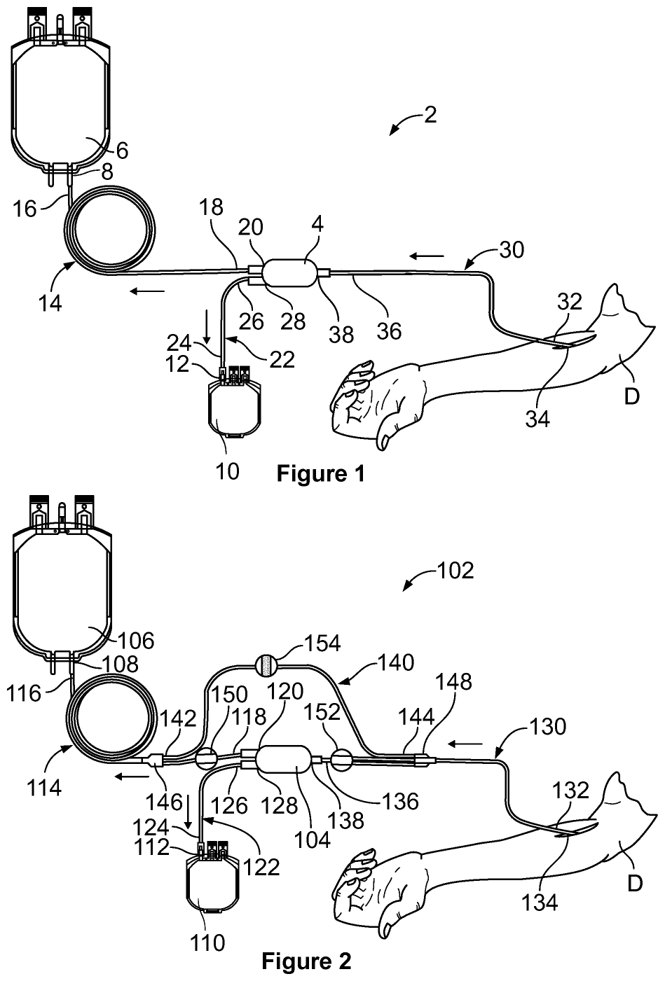

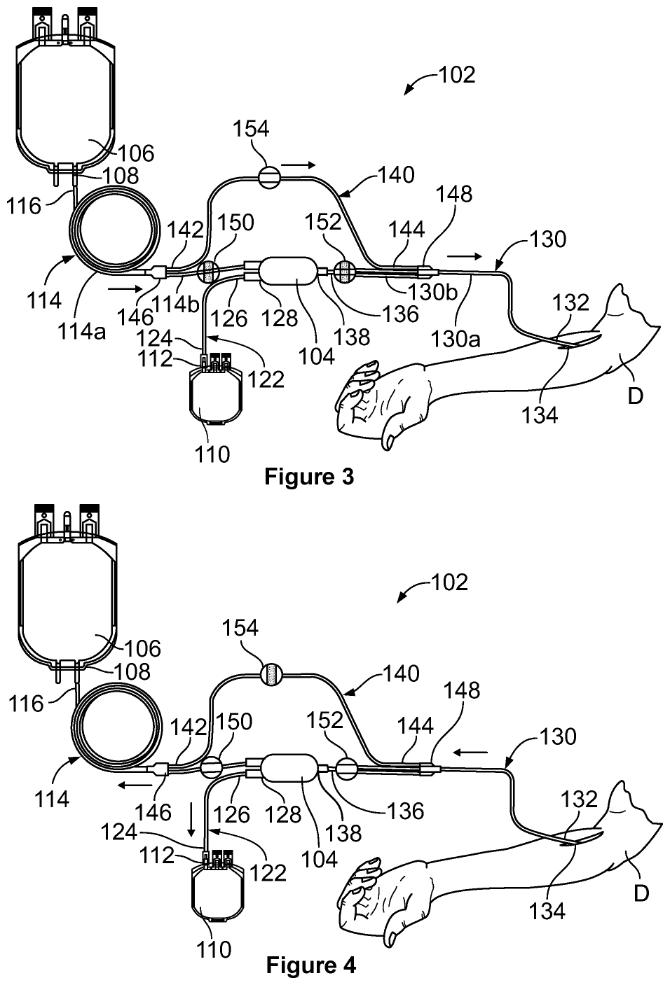

FIGS. 1-4 illustrate two example embodiments of such blood pack donation systems. The first embodiment, shown in FIG. 1, provides for a single pass collection procedure via connection to a LOC device, which permits collecting WB during a WB donation, while screening for and collecting biomarkers. The second embodiment, shown in FIGS. 2-4, provides for a single pass collection procedure or an optional multiple pass collection procedure via connection to a LOC device. The second embodiment includes a plurality of flow control components wherein a single pass collection procedure comparable to that of the first embodiment may be performed with the plurality of flow control components having first selected settings. The second embodiment permits a multiple pass collection procedure that may be performed with the plurality of flow control components having a succession of further second and third selected settings, and which permits collecting WB during a WB donation, while screening for and collecting biomarkers.

Turning now to FIG. 1, a blood pack donation system 2 is configured for use with a LOC device 4 for biomarker collection during WB donation. Thus, the blood pack donation system 2 preferably is presterilized and configured for use with any of a variety of LOC devices 4, which may be provided by various manufacturers for use in separating and screening for collection any of a number of different biomarkers. Thus, makers of LOC devices may construct or integrate their LOC devices for use with the system 2.

The system 2 includes a blood collection container 6 having a volume and at least one opening 8, and a biomarker collection container 10 having a volume and at least one opening 12. A first flow path 14 having a first end 16 is connected to the opening 8 in the blood collection container 6 and a second end 18 is configured to be connected to a first outlet opening 20 of the LOC device 4. A second flow path 22 having a first end 24 is connected to the opening 12 in the biomarker collection container 10 and a second end 26 configured to be connected to a second outlet opening 28 of the LOC device 4. A third flow path 30 having a first end 32 is connected to a needle 34 and a second end 36 configured to be connected to an inlet opening 38 of the LOC device 4. The needle 34 is configured for use with a donor D, for use in the manner of a routine WB donation procedure.

As noted above, the respective openings in the blood collection container 6 and biomarker collection container 10 may be preexisting or formed when connected to a respective flow path 16, 22. In addition, the flow paths may be constructed in the form of individual sections of tubing, or may be integrated into a cassette, whether in the form of channels or sections of tubing. It will be appreciated that the first example system 2 may have an alternative configuration in the form of a cassette, which may provide the respective flow paths already connected to a housing, which may be represented by the body 4, which in turn may accept a LOC device for connection to the respective flow paths for use.

As previously noted, the blood pack donation system 2 preferably is presterilized. The system 2 may be configured with integrated fixed connections between all or some of the components, such as via heat sealing, solvent bonding or other suitable means of connection. Connectors may be used to permanently join separated components. In a further alternative, all or some of the connections may be removably connected, such as by means of sterile unions using Luer-Lock connectors, or other suitable connection devices.

For example, the opening 8 of the blood collection container 6 may be removably connected to the first end 16 of the first flow path 14, and the first outlet opening 20 of the LOC device 4 may be removably connected to the second end 18 of the first flow path 14. Similarly, the opening 12 of the biomarker collection container 10 may be removably connected to the first end 24 of the second flow path 22, and the second outlet opening 28 of the LOC device 4 may be removably connected to the second end 26 of the second flow path 22. In turn, the needle 34 may be removably connected to the first end 32 of the third flow path 30, and the inlet opening 38 of the LOC device 4 may be removably connected to the second end 36 of the third flow path 30.

It will be appreciated that the system 2 may include fixed connections between components, but the system 2 may be configured for connection to the LOC device 4 at the point of use, so removable connections may be provided at least at the second end 18 of the first flow path 14, at the second end 26 of the second flow path 22 and at the second end 36 of the third flow path 30, for connection respectively to the LOC device 4 first outlet opening 20, second outlet opening 28 and inlet opening 38. Alternatively, manufacturers of LOC devices may fixedly connect such devices to the system 2, if desired.

Turning to FIGS. 2-4, a second example blood pack donation system 102 is configured for use with a LOC device 104 for biomarker collection during WB donation. The LOC device 104 may be similar to the LOC device 4 of the first example. The blood pack donation system 102 also preferably is presterilized and configured for use with any of a variety of LOC devices 104, from various manufacturers and for use in separating and screening for collection any of a number of different biomarkers. Accordingly, makers of LOC devices may construct or integrate their LOC devices for use with the system 102.

The system 102 includes a blood collection container 106 having a volume and at least one opening 108, and a biomarker collection container 110 having a volume and at least one opening 112. A first flow path 114 having a first end 116 is connected to the opening 108 in the blood collection container 106 and a second end 118 is configured to be connected to a first outlet opening 120 of the LOC device 104. A second flow path 122 having a first end 124 is connected to the opening 112 in the biomarker collection container 110 and a second end 126 configured to be connected to a second outlet opening 128 of the LOC device 104. A third flow path 130 having a first end 132 is connected to a needle 134 and a second end 136 configured to be connected to an inlet opening 138 of the LOC device 104. The needle 134 is configured for use with a donor D, for use in the manner of a routine WB donation procedure.

The second example system 102 further includes a fourth flow path 140 having a first end 142 connected to the first flow path 114 at a location between the first end 116 and second end 118 of the first flow path 114 and having a second end 144 connected to the third flow path 130 at a location between the first end 132 and second end 136 of the third flow path 130. The connections at the first end 142 and second end 144 of the fourth flow path 140 are shown in FIGS. 2-4 as Y-connectors 146 and 148, but are considered herein to be connections along lengths of the first flow path 114 and the third flow path 130. Consistent with the above discussion of the first example system 2, the flow path connections in the second example system 102 may be integrated or fixed, or removably connected, and the respective openings in the blood collection container 106 and biomarker collection container 110 may be preexisting or formed when connected to a respective flow path 116, 122. In addition, the flow paths may be constructed in the form of individual sections of tubing, or may be integrated into a cassette, whether in the form of channels or sections of tubing. As noted with respect to the first example system 2, a further alternative configuration of the second example system 102 may be in the form of a cassette, which may provide the respective flow paths already connected to a housing, which may be represented by the body 104, which in turn may accept a LOC device for connection to the respective flow paths for use.

Thus, similarly to the first example system 2, in the second system 102 the opening 108 of the blood collection container 106 may be removably connected to the first end 116 of the first flow path 114, and the first outlet opening 120 of the LOC device 104 may be removably connected to the second end 118 of the first flow path 114. Similarly, the opening 112 of the biomarker collection container 110 may be removably connected to the first end 124 of the second flow path 122, and the second outlet opening 128 of the LOC device 104 may be removably connected to the second end 126 of the second flow path 122. In turn, the needle 134 may be removably connected to the first end 132 of the third flow path 130, and the inlet opening 138 of the LOC device 104 may be removably connected to the second end 136 of the third flow path 130. The first end 142 of the fourth flow path 140 may be removably connected to the first flow path 114 and the second end 144 of the fourth flow path 140 may be removably connected to the third flow path 130.

As with the previous example, removable connections may be provided in the second example system 102 at least at the second end 118 of the first flow path 114, at the second end 126 of the second flow path 122 and at the second end 136 of the third flow path 130, for connection respectively to the first outlet opening 120, second outlet opening 128 and inlet opening 138 of the LOC device 104. Alternatively, manufacturers of LOC devices may fixedly connect such devices to the system 102, if desired.

The second example blood pack donation system 102 further includes a plurality of flow control components 150, 152 and 154 that selectively control flow through the first flow path 114, third flow path 130 and fourth flow path 140, respectively. The plurality of flow control components 150, 152 and 154 that selectively control flow through the first flow path 114, third flow path 130 and fourth flow path 140 is selectively adjustable for use with a LOC device 104 to provide a single pass collection procedure or a multiple pass collection procedure for collecting WB during a WB donation, while also collecting biomarkers.

The second example system 102 optionally is capable of use in a multiple pass collection procedure, which includes a succession of adjustments of the plurality of flow control components 150, 152 and 154. For example, as shown in FIG. 2, the plurality of flow control components 150, 152 and 154 is adjusted in a first step to permit flow through the first flow path 114 to the blood collection container 106 and through the third flow path 130 to the LOC device 104, while preventing flow through the fourth flow path 140. With such settings of the flow control components, WB may flow through the third flow path 130, the LOC device 104, the first flow path 114 to collect WB in the WB collection container 106, while biomarkers flow through the second flow path 122 and are collected in the biomarker collection container 110. It will be appreciated that in step one, the flow control component 154 may partially or completely close the fourth flow path 140, so as to permit much or most of the WB to flow from the donor D to the blood collection container 106, while a lesser portion of the WB from the donor D must pass through the LOC device 104. This may permit a more rapid procedure, while still providing screening and collection of biomarkers from an adequate portion of the WB donation. Similarly, the flow control component 152 optionally may utilize a setting that selectively reduces or increases the flow through the LOC device 104, independently of the WB flow through the fourth flow path 140.

With the system 102, in a second step of a multiple pass collection procedure, the plurality of flow control components 150, 152 and 154 is further adjustable to permit reverse flow through a first portion 114a of the first flow path 114, through the fourth flow path 140 and through and a first portion 130a of the third flow path 130, while preventing reverse flow through a second portion 114b of the first flow path 114 and a second portion 130b of the third flow path 130. For example, this may be accomplished if the flow control component 154 permits the fourth flow path 140 to be partially or fully open, while the flow control components 150 and 152 fully close the second portion 114b of the first flow path 114 and the second portion 130b of the third flow path 130.

To complete the multiple pass collection procedure with the second example system 102, in a third step the plurality of flow control components 150, 152 and 154 is further adjustable and may be similar to the first step, so as to permit at least some flow through the first flow path 114 to the blood collection container 106 and through the third flow path 130 to the LOC device 104, while partially or fully preventing flow through the fourth flow path 140. Thus, WB may flow through the third flow path 130, the LOC device 104, the first flow path 114 to collect WB in the WB collection container 106, while biomarkers flow through the second flow path 122 and are collected in the biomarker collection container 110.

It will be appreciated that the plurality of flow control components 150, 152 and 154 further may include a plurality of respective clamps, such as Roberts-type clamps, pinch, slide or roller clamps, or other suitable types of clamps usable to selectively prevent or permit flow through a flow path that may be a section of tubing or a channel in a cassette or other suitable structure. Accordingly, the plurality of respective clamps 150, 152 and 154 is adjustable with respect to flow through the first flow path 114 to the blood collection container 106 and through the third flow path 130 to the LOC device 104, as well as the flow through the fourth flow path 140, for use in a single pass collection procedure or optionally in a multiple pass collection procedure.

Consistent with the above description, the system 102 may be further adjusted for a second step of a multiple pass collection procedure wherein the plurality of respective clamps 150, 152 and 154 is further adjustable to permit reverse flow from the blood collection container 106 through a first portion 114a of the first flow path 114, through the fourth flow path 140 and through a first portion 130a of the third flow path 130, while preventing reverse flow through a second portion 114b of the first flow path 114 and a second portion 130b of the third flow path 130.

It will be appreciate that in the second step of the multiple pass collection procedure, to prevent reverse flow through the LOC device 104, one of the respective clamps 152 is adjustable to selectively prevent reverse flow through the third flow path 130 at a location between the second end 136 of the third flow path 130 and the connection of the fourth flow path 140 to the third flow path 148. A further one of the respective clamps 150 is adjustable to selectively prevent reverse flow through the first flow path 114 at a location between the second end 118 of the first flow path 114 and the connection of the fourth flow path 140 to the first flow path 146.

Accordingly, example blood pack donation systems configured for use with a lab-on-a-chip device for biomarker collection during whole blood donation are disclosed and may be used to conduct a single pass collection procedure or a multiple pass collection procedure.

It will be understood that the embodiments and examples described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

### Other Aspects

Aspect 1. A blood pack donation system configured for use with a lab-on-a-chip device for biomarker collection during whole blood donation comprising: a blood collection container having a volume and at least one opening; a biomarker collection container having a volume and at least one opening; a first flow path having a first end connected to the opening in the blood collection container and a second end configured to be connected to a first outlet opening of a lab-on-a-chip device; a second flow path having a first end connected to the opening in the biomarker collection container and a second end configured to be connected to a second outlet opening of the lab-on-a-chip device; and a third flow path having a first end connected to a needle and a second end configured to be connected to an inlet opening of the lab-on-a-chip device.

Aspect 2. The blood pack donation system of Aspect 1 wherein the opening of the blood collection container is removably connected to the first end of the first flow path.

Aspect 3. The blood pack donation system of Aspect 2 wherein the first outlet opening of the lab-on-a-chip device is removably connected to the second end of the first flow path.

Aspect 4. The blood pack donation system of Aspect 1 wherein the opening of the biomarker collection container is removably connected to the first end of the second flow path.

Aspect 5. The blood pack donation system of Aspect 4 wherein the second outlet opening of the lab-on-a-chip device is removably connected to the second end of the second flow path.

Aspect 6. The blood pack donation system of Aspect 1 wherein the needle is removably connected to the first end of the third flow path.

Aspect 7. The blood pack donation system of Aspect 6 wherein the inlet opening of the lab-on-a-chip device is removably connected to the second end of the third flow path.

Aspect 8. The blood pack donation system of Aspect 1 further comprising a fourth flow path having a first end connected to the first flow path at a location between the first end and second end of the first flow path and having a second end connected to the third flow path at a location between the first end and second end of the third flow path.

Aspect 9. The blood pack donation system of Aspect 8 wherein the first end of the fourth flow path is removably connected to the first flow path and the second end of the fourth flow path is removably connected to the third flow path.

Aspect 10. The blood pack donation system of Aspect 9 further comprising a plurality of flow control components that selectively control flow through the first flow path, third flow path and fourth flow path.

Aspect 11. The blood pack donation system of Aspect 10 wherein the plurality of flow control components that selectively control flow through the first flow path, third flow path and fourth flow path is selectively adjustable for use with a lab-on-a-chip device to provide a single pass collection procedure or a multiple pass collection procedure for collecting whole blood during a whole blood donation, while also collecting biomarkers.

Aspect 12. The blood pack donation system of Aspect 11 wherein the multiple pass collection procedure includes a succession of adjustments of the plurality of flow control components.

Aspect 13. The blood pack donation system of Aspect 11 wherein the plurality of flow control components is adjustable to permit flow through the first flow path to the blood collection container and through the third flow path to the lab-on-a-chip device, while permitting partial flow or preventing flow through the fourth flow path.

Aspect 14. The blood pack donation system of Aspect 13 wherein the plurality of flow control components is further adjustable to permit reverse flow through a first portion of the first flow path, through the fourth flow path and through and a first portion of the third flow path, while preventing reverse flow through a second portion of the first flow path and a second portion of the third flow path.

Aspect 15. The blood pack donation system of Aspect 14 wherein the plurality of flow control components is further adjustable to permit flow through the first flow path to the blood collection container and through the third flow path to the lab-on-a-chip device, while permitting partial flow or preventing flow through the fourth flow path.

Aspect 16. The blood pack donation system of Aspect 10 wherein the plurality of flow control components further comprises a plurality of respective clamps.

Aspect 17. The blood pack donation system of Aspect 16 wherein the plurality of respective clamps is adjustable to permit flow through the first flow path to the blood collection container and through the third flow path to the lab-on-a-chip device, while permitting partial flow or preventing flow through the fourth flow path.

Aspect 18. The blood pack donation system of Aspect 17 wherein the plurality of respective clamps is further adjustable to permit reverse flow from the blood collection container through a first portion of the first flow path, through the fourth flow path and through a first portion of the third flow path, while preventing reverse flow through a second portion of the first flow path and a second portion of the third flow path.

Aspect 19. The blood pack donation system of Aspect 18 wherein one of the respective clamps is adjustable to selectively prevent reverse flow through the third flow path at a location between the second end of the third flow path and the connection of the fourth flow path to the third flow path.

Aspect 20. The blood pack donation system of Aspect 19 wherein a further one of the respective clamps is adjustable to selectively prevent reverse flow through the first flow path at a location between the second end of the first flow path and the connection of the fourth flow path to the first flow path.

1. A blood pack donation system configured for use with a lab-on-a-chip device for biomarker collection during whole blood donation comprising:
a) a blood collection container having a volume and at least one opening;
b) a biomarker collection container having a volume and at least one opening;
c) a first flow path having a first end connected to the opening in the blood collection container and a second end configured to be connected to a first outlet opening of a lab-on-a-chip device;
d) a second flow path having a first end connected to the opening in the biomarker collection container and a second end configured to be connected to a second outlet opening of the lab-on-a-chip device; and
e) a third flow path having a first end connected to a needle and a second end configured to be connected to an inlet opening of the lab-on-a-chip device.

2. The blood pack donation system of claim 1 wherein the opening of the blood collection container is removably connected to the first end of the first flow path.

3. The blood pack donation system of claim 2 wherein the first outlet opening of the lab-on-a-chip device is removably connected to the second end of the first flow path.

4. The blood pack donation system of claim 1 wherein the opening of the biomarker collection container is removably connected to the first end of the second flow path.

5. The blood pack donation system of claim 4 wherein the second outlet opening of the lab-on-a-chip device is removably connected to the second end of the second flow path.

6. The blood pack donation system of claim 1 wherein the needle is removably connected to the first end of the third flow path.

7. The blood pack donation system of claim 6 wherein the inlet opening of the lab-on-a-chip device is removably connected to the second end of the third flow path.

8. The blood pack donation system of claim 1 further comprising a fourth flow path having a first end connected to the first flow path at a location between the first end and second end of the first flow path and having a second end connected to the third flow path at a location between the first end and second end of the third flow path.

9. The blood pack donation system of claim 8 wherein the first end of the fourth flow path is removably connected to the first flow path and the second end of the fourth flow path is removably connected to the third flow path.

10. The blood pack donation system of claim 9 further comprising a plurality of flow control components that selectively control flow through the first flow path, third flow path and fourth flow path.

11. The blood pack donation system of claim 10 wherein the plurality of flow control components that selectively control flow through the first flow path, third flow path and fourth flow path is selectively adjustable for use with a lab-on-a-chip device to provide a single pass collection procedure or a multiple pass collection procedure for collecting whole blood during a whole blood donation, while also collecting biomarkers.

12. The blood pack donation system of claim 11 wherein the multiple pass collection procedure includes a succession of adjustments of the plurality of flow control components.

13. The blood pack donation system of claim 11 wherein the plurality of flow control components is adjustable to permit flow through the first flow path to the blood collection container and through the third flow path to the lab-on-a-chip device, while permitting partial flow or preventing flow through the fourth flow path.

14. The blood pack donation system of claim 13 wherein the plurality of flow control components is further adjustable to permit reverse flow through a first portion of the first flow path, through the fourth flow path and through and a first portion of the third flow path, while preventing reverse flow through a second portion of the first flow path and a second portion of the third flow path.

15. The blood pack donation system of claim 14 wherein the plurality of flow control components is further adjustable to permit flow through the first flow path to the blood collection container and through the third flow path to the lab-on-a-chip device, while permitting partial flow or preventing flow through the fourth flow path.

16. The blood pack donation system of claim 10 wherein the plurality of flow control components further comprises a plurality of respective clamps.

17. The blood pack donation system of claim 16 wherein the plurality of respective clamps is adjustable to permit flow through the first flow path to the blood collection container and through the third flow path to the lab-on-a-chip device, while permitting partial flow or preventing flow through the fourth flow path.

18. The blood pack donation system of claim 17 wherein the plurality of respective clamps is further adjustable to permit reverse flow from the blood collection container through a first portion of the first flow path, through the fourth flow path and through a first portion of the third flow path, while preventing reverse flow through a second portion of the first flow path and a second portion of the third flow path.

19. The blood pack donation system of claim 18 wherein one of the respective clamps is adjustable to selectively prevent reverse flow through the third flow path at a location between the second end of the third flow path and the connection of the fourth flow path to the third flow path.

20. The blood pack donation system of claim 19 wherein a further one of the respective clamps is adjustable to selectively prevent reverse flow through the first flow path at a location between the second end of the first flow path and the connection of the fourth flow path to the first flow path.
